(19) United States
(12) Patent Application Publication
(10) Pub. No.: US 2004/0157788 A1
(43) Pub. Date: Aug. 12, 2004

(54) High affinity antibodies

(76) Inventor: Peter John Harrison, Esher (GB)

Correspondence Address: Testa Hurwitz & Thibeault, Patent Administrator, High Street Tower, 125 High Street, Boston, MA, 02110, US

(21) Appl. No.: 10/451,302

(22) PCT Filed: Dec. 21, 2001
(86) PCT No.: PCT/GB01/05759

(30) Foreign Application Priority Data
Dec. 21, 2000 (GB) 0031284.3

(51) Int. Cl. A61K 48/00; A61K 39/395; C12N 5/06

(52) U.S. Cl. 514/44; 536/23.53; 435/326

(57) ABSTRACT

Methods for selecting high affinity monoclonal antibodies are provided with gene loci for expressing such high affinity antibodies.

### FIELD OF THE INVENTION

This invention relates to monoclonal antibodies having high affinity as well as to methods for identifying/producing such antibodies.

### BACKGROUND OF THE INVENTION

Murine and rat monoclonal antibodies are widely available, but are generally unsuitable for therapeutic use in humans owing to the human antibody response to foreign proteins. In order to overcome this problem, it has been proposed to chimerise rodent monoclonals, by combining the variable region of the monoclonal with the constant region derived from human immunoglobulin. In order to produce suchmonoclonal antibodies in bulk, the gene for a chimeric or other antibody of this type can be transfected into and expressed by myeloma cells or any other host cell line.

Another disadvantage associated with rodent antibodies is their low affinity. The affinity constant (Ka) of a murine monoclonal is typically no more than 1010. Humanisation will often reduce the Ka value, although the original value may be substantially retained, by careful choice of the materials and experimental conditions. This has generally been the approach to the high production of high affinity monoclonals intended for therapeutic use in humans.

Groves et al, J. Endocrinol. 126 (1990) 217-222, describe a stable high-affinity ovine monoclonal against progesterone following fusion with the murine NS1 myeloma. WO-A-92/15699 describes the Humanisation of antibodies having high affinity, including ovine monoclonal antibodies. QW5770429 discloses transgenic mice that produce human antibodies, wherein the human heavy chain and human light chain are encoded by a transgene stably integrated into the mouse cell genome. The transgenic mouse is then capable of producing immunoglobulin chains that are encoded by human immunoglobulin genes, and is capable of eliciting an immune response against human antigens. However, these antibodies have relatively low affinity.

For the production of an antibody, a gene locus (or ‘mini-locus’) may be provided, comprising genes for V and J regions, in clusters, in one host. On expression, these regions can combine in various ways, producing many functional antibodies.

### SUMMARY OF THE INVENTION

The present invention is based on the discovery of a common feature among monoclonal antibodies having high affinity. Although more detailed results are presented below, it has surprisingly been found that utilisation of a λ light chain in a monoclonal antibody is desirable, in that it is associated with high affinity. Accordingly, although antibodies having A light chains are known, it has now been appreciated that selecting for such antibodies is useful. Such selection may be achieved in a variety of ways.

According to a first aspect of the invention, there is provided a gene locus comprising a plurality of genes encoding at least the variable region of an antibody light chain, joining regions, the variable regions of the heavy chain and optionally the constant region, wherein the light chain regions encoded have the sequence or other characteristic of λ light chain.

A gene locus of the invention may be provided and expressed in a suitable host. A method for the production of a monoclonal antibody or fragment thereof, comprises expressing the genes as defined above, and optionally also selecting the products of recombination.

According to a further aspect of the invention, there is provided a method for modifying cells so that they are capable of producing a monoclonal antibody or fragment thereof, comprises inactivating the host's machinery for producing antibodies, if present, and introducing genes as defined above. Typically, the monoclonal antibody or fragment thereof, comprises a λ light chain, a heavy chain and, optionally, a human constant region, and wherein at least the variable regions of the light chains have the sequence or other characteristic of λ light chains.

According to yet another aspect of the invention, there is provided a library of monoclonal antibodies or fragments thereof, wherein the light chains have the sequence or other characteristics of a λ light chain. The library may be displayed in phage or another suitable host.

The present invention may be used to produce antibodies with high affinity. Particular advantages of high affinity antibodies, e.g. for use in therapy, are:

(i) Affinity is related to biological response. The higher the affinity, the better the response is likely to be.

(ii) Higher affinity antibodies will result in more rapid binding of the antibodies to the target cells or more rapid immunoneutralisation. This means that there will be better localisation of antibody or antibody conjugate.

(iii) Higher affinity means that fewer antibodies can be used per dosage, leading to more economically viable therapies. This is a relevant consideration where competition reactions are involved, e.g. binding with a virus or lymphokine rather than the virus or lymphokine binding with a cell surface or receptor.

In yet a further aspect, the invention provides a method for selecting high affinity monoclonal antibodies which comprises the step of screening one or more monoclonal antibodies and selecting those which have λ light chains. In yet another aspect, the invention provides the use of a monoclonal antibody produced and/or identified according to the methods of the present invention in medicine.

### DESCRIPTION OF THE INVENTION

The present invention involves avoidance of the production of antibodies containing K light chains, in order to provide high affinity reproducibly. The antibody may be chimeric, combining a non-human λ\ light chain locus for the variable region, i.e. V and J regions, together with human constant region (for either λ or K) together with relevant heavy chain machinery. Alternatively, the antibody may be fully human, using the human λ light chain locus for the variable region (V and J regions) together with human constant region (for either λ\ or K) together with relevant heavy chain machinery.

An aspect of this invention lies in antibody libraries where light chain variable regions are λ. The λ variable regions together with relevant heavy chain genes may be displayed in, for example, in an antibody library system, phage or ribosome.

Typically, an antibody of the present invention has an affinity of at least 10¹⁰ l/mol, preferably at least 10¹¹ l/mol, more preferably at least 10¹² l/mol, and most preferably at least 10¹³ l/mol. The affinity may be as high as 10¹⁵ l/mol.

In a preferred embodiment of the invention, the antibodies are produced by first creating a transgenic animal that incorporates a gene locus according to the invention.

Transgenic animals suitable to produce the antibodies may be developed using techniques known in the art. In particular, U.S. Pat. Nos. 5,770,429 and 5,545,807 describe how to design suitable transgenes for insertion into an animal's cell genome. This technique may be adapted using the gene loci as defined herein as the transgene. The transgenes integrated into the animal must function correctly throughout the pathway of B-cell development. Typically the transgene will comprise DNA encoding at least one human Ig heavy chain and at least one human Ig light chain. U.S. Pat. No. 5,770,429 details the construction of “mini-loci” containing multiple V, D and J gene segments, μC_{H} segment, at least one additional gene segment for a heavy chain constant region, together with all the necessary sequences needed for isotype switching. Again, this may be adapted with the design of gene loci having λ light chains. It is preferable that the transgene contain multiple copies of the gene segments which are operably linked and capable of functional rearrangement as this results in a much wider generation of diversity in the antibody response.

There are examples in the art of the production of constructs for producing suitable antibodies. These include Popov A V, Zou X, Xian J, Nicholson I C, Br\ddot{ua}ggemann M J Exp Med 1999 May 189:1611-20 and Popov A V, Bützler C, Frippiat J P, Lefranc M P, Br\ddot{ua}ggemann M, Gene 1996 Oct 177:195-201. After preparation of suitable transgenes, the transgenic animal can be produced by introducing a suitable transgene into the germ line of a non-human, animal. Methods suitable for introducing the transgene into the animal's germ line, e.g. into an early embryo, are known to those skilled in the art, for example, transgene microinjection into early totipotent, embryonic cells.

Existing cloning techniques may also be used, and this may provide better results for large animals. This involves manipulation at the zygote level and cloning the DNA of e.g. fibroblasts, using a one step method to replace the endogenous immunoglobulin genes with the human equivalent. There are several advantages to this; the method is faster and does not necessitate breeding which often has poor results and may put the animals with immunoglobulin knockouts at risk.

The transgenes may also be incorporated by homologous recombination, as detailed in Bradley, Current Opinion in Biotechnology, 1991; 2:823-829.

In producing the transgenic animals, it may be desirable to disrupt the endogenous immunoglobulin locus of the animal to render it B-cell deficient. This is reported by Wagner et al, Nucleic Acids Research, 1994; 22(8):1389-1393, to improve antibody production by the incorporated transgenes. Methods to disrupt the endogenous immunoglobulin loci in the animal will be apparent to the skilled person. In one example, the transgene itself may be constructed to target the loci, to disrupt endogenous production.

The transgenic animal may be a rodent, or more preferably a larger animal which may help produce high affinity antibodies. Suitable animals are rabbits, donkeys, goat, cows and sheep. The use of sheep exploits the inter-species differences in cellular and molecular mechanisms for generating a functional B-cell repertoire (Weill and Reynaud, 1992). In the mouse, the B-cell precursors undergo recombination in the bone marrow, which generates immunoglobulin gene combinations. In sheep (ruminants), primary B-cell diversity is accomplished in gut-associated lymphoid tissue by recombination and somatic cell hypermutation (Weill and Raynaud 1992, Dufour et al 1996). This difference may account for the higher affinity antibodies produced by sheep and may also be important for the immune system's ability to recognise and respond to antigens. It has been noted that the injection of the sample complex antigen (for example, multiple proteins) into both mouse and sheep will generate not only a different immunoglobulin response in regard to the proteins/epitopes recognised, but the sheep will respond to a broader range of epitopes. This function becomes commercially valuable in the field of anti-cancer antibodies where the researcher is highly dependent on the immune system's ability to generate an immunoglobulin response against a cancer-specific antigen.

Various techniques can be applied, in order to produce antibodies in accordance with the present invention. In a preferred embodiment, the product is, or is predominantly, of human origin, allowing it to be used satisfactorily in a method of therapy practiced on a human, but the relative proportions of human immunoglobulin in the product will depend on the method by which it is prepared.

A whole antibody comprises heavy and light chains, and constant and variable regions. The variable regions comprise a variable framework and hypervariable regions within which the antigen-binding sites are located. The invention includes antibody fragments within its scope, e.g. F(ab′)₂, Fab or Fv fragments, provided that the light chain is a λ light chain. Preferably, at least part of the light chain is derived from human immunoglobulin genes. More preferably, an antibody of the invention will comprise both variable and constant regions encoded by human Ig genes.

Antibodies may be prepared by a process involving essentially two steps. Firstly, a suitable transgenic animal is immunised using an antigen, and B cells secreting an antibody to that antigen are obtained. Secondly, high-affinity monoclonal antibodies and the genes coding for them are obtained by conventional hybridoma technology, followed by gene cloning and expression of the human immunoglobulin in an appropriate cell line containing an appropriate expression system. Those antibodies having λ light chains are for use in this invention.

Hybridoma technology comprises the fusion of the B cells secreting high-affinity human antibodies with myeloma cells, and selection of resultant hybridomas. Alternatively, the B cells from the relevant species can be plated out, selected and amplified, e.g. by using the polymerase chain reaction; phage recovery may also be used, to obtain mRNA from selected lymphocytes, and thus the antibody genes.

In generating the high affinity monoclonals by hybridoma technology, a suitable fusion partner should be selected. If sheep are used as the transgenic animal, it is possible to prepare the hybridomas using SFP1, a sheep-mouse heteromyeloma fusion partner derived originally from the NS1 cell line.

Methods for screening monoclonal antibodies for the presence of λ or κ light chains are well known to those skilled in the art. For instance ELISA based methods can be used utilising anti-lambda antibodies. Thus, the antigen is first immobilised on a surface, such as a plate, to which is added the monoclonal antibody to be screened, followed by the anti-lambda antibody. The anti-lambda antibody may be conjugated to an enzyme such as alkaline phosphatase, which allows detection using a standard enzyme assay. Alternatively, an unconjugated anti-lambda antibody can be used together with an additional antibody-enzyme conjugate. Examples of suitable reagents include Sigma A2904, which is an anti-human lambda light chain antibody conjugated to alkaline phosphatase or VRMD Inc's BIG501E, which is an anti-ovine/bovine lambda light chain antibody. In the case of the latter it would be necessary to use an anti-murine antibody conjugated to an enzyme such as alkaline phosphatase.

An antibody of the invention may be used in therapy. It is likely to be of particular value in the treatment of cancer, inflammation, e.g. rheumatoid arthritis, and septic shock, following organ transplant, in immunomodulation, for passive immunotherapy in the treatment of viruses, and to provide antibodies against bacteria. For this purpose, the antibody may be formulated into a suitable composition with a physiologically-acceptable excipient diluent or carrier. Formulating a suitable composition for delivery to a patient will be apparent to the skilled person. Delivery of antibodies is also well established and again will be apparent to the skilled person.

The evidence on which the present invention is based will now be described.

Hybridoma fusions have been performed using standard hetero-hybridoma techniques and screened for monoclonals of higher than average (i.e. with respect to the polyclonal population) affinity by acid washed ELISA. Light chains were isolated by PCR cloning techniques, using primers designed for sheep λ and K light chain sequences.

The following Table shows the results that were found. They indicate that a high acid wash value (low pH, correlates with high affinity) is associated in all cases with the presence of λ light chains.

TABLE

| Antigen | Clone | PH50% | Light Chain |
|---|---|---|---|
| CEA | polyclonal | 3.4 | mix |
| | 6H9 | 1.8 | lambda |
| EGFr | polyclonal | 3.4 | mix |
| | 1B5 | 2.3 | lambda |
| VEGF | polyclonal | 3.7 | mix |
| | 1F12 | 3.0 | lambda |
| T3 | polyclonal | 2.7 | mix |
| | 17C6 | 2.0 | lambda |
| TNF | Polyclonal | 3.7 | mix |
| | 5D10 | 2.2 | lambda |
| | 5C6 | 2.5 | Lambda |

1. A gene locus comprising a plurality of genes encoding at least the variable regions of an antibody light chain, joining regions, the variable regions of the heavy chain and optionally constant regions wherein the light chain regions encoded have the sequence or other characteristic of λ light chain.

2. A host comprising the genes defined in claim 1, which is capable of expressing the genes.

3. Use of a gene locus according to claim 1, for the transfection of a host cell.

4. A method for the production of a monoclonal antibody or fragment thereof, which comprises expressing the genes as defined in claim 1, and optionally also selecting the products of recombination.

5. A method for modifying cells so that they are capable of producing a monoclonal antibody or fragment thereof, which comprises inactivating the host's machinery for producing antibodies, if present, and introducing genes as defined in claim 1.

6. A method according to claim 4 or claim 5, wherein the monoclonal antibody or fragment thereof, comprises a light chain and a heavy chain, and wherein constant region may be of human origin, and wherein at least the variable regions of the light chains have the sequence or other characteristic of λ light chains.

7. A method according to any of claims 4 to 6, wherein the antibody has an affinity of at least 10¹⁰ l/mol.

8. A method according to claim 7, wherein the antibody has an affinity of at least 10¹³ l/mol.

9. A method according to any of claims 4 to 8, wherein the antibody or fragment comprises a Fab or Fv fragment.

10. A library of monoclonal antibodies or fragments thereof, wherein at least the light chains have the sequence or other characteristic of λ light chains.

11. A library according to claim 10, which comprises fragments that are λ light chains.

12. A library according to claim 10 or claim 11, wherein the antibodies are humanised or human.

13. A library according to any of claims 10 to 12, wherein the antibodies have one or more characteristics as defined in claims 6 to 9.

14. A host displaying a library according to any of claims 10 to 13.

15. A method for selecting high affinity monoclonal antibodies which comprises the step of screening one or more monoclonal antibodies and selecting those which have λ light chains.

16. The use of a monoclonal antibody produced and/or identified according to any one of claims 4 to 9 in medicine.
